(19) United States
(12) Patent Application Publication
(10) Pub. No.: US 2026/0221276 A1
(43) Pub. Date: Jul. 30, 2026

(54) Installation Requirement Determination for a Medical Floor-Mounted Device

(71) Applicant: Siemens Healthineers AG, Forchheim (DE)

(72) Inventors: Thomas Beck, Dormitz (DE); Jeffrey Lyness, Erlangen (DE); Stephan Biber, Erlangen (DE)

(73) Assignee: Siemens Healthineers AG, Forchheim (DE)

(21) Appl. No.: 19/464,745

(22) Filed: Jan. 30, 2026

(30) Foreign Application Priority Data
Jan. 30, 2025 (DE) 10 2025 103 439.7

(51) Int. Cl. G16H 40/40

(52) U.S. Cl. CPC G16H 40/40

(57) ABSTRACT

A computer-implemented method for a medical floor-mounted device, including: providing a digital model of a medical floor-mounted device, the digital model having system information of the medical floor-mounted device which includes at least component information regarding system components of the medical floor-mounted device; and at least one item of object information including at least one spatial requirement of a system component of the medical floor-mounted device, the at least one item of object information being associated with at least one item of system information; and deriving from the digital model of minimum distances that must be taken into consideration when installing the medical floor-mounted device.

### TECHNICAL FIELD

The present disclosure relates to a computer-implemented method for a medical floor-mounted device, a computer program, and a digital storage medium.

### BACKGROUND

Independent of the grammatical term usage, individuals with male, female, or other gender identities are included within the term.

Medical floor-mounted devices, such as magnetic resonance imaging (MRI) systems, are relatively large and heavy and are often set up in specially dedicated rooms. When planning rooms for such medical devices, certain minimum distances to the side walls, the ceiling, and the floor of the room must be observed for operation, service, maintenance, repair, or replacement of components. For example, for the replacement of certain system components, in each case, a specific amount of space is required next to and/or above the device in order to remove and reinstall the components. Each model of a medical floor-mounted device has different requirements in this regard. Each model has various components, various options for the various components, and various positioning options for integrating the respective component into the respective option in the medical floor-mounted device. This results in a multiplicity of possible combinations for each model in a multiplicity of models, e.g., more than 500 possible combinations.

In addition, there are continuous developments of medical floor-mounted devices. Individual parts or system components may be developed. This may also lead to changes in the dimensions and distances of medical floor-mounted devices and/or individual components and/or the interaction of individual components.

This multiplicity of possible combinations and the constant changes to at least individual components or system components make it impossible even for experienced engineers and/or developers as well as service and maintenance personnel to be familiar with or have an overview of the respective dimensions and distances of all models in all variations. Accordingly, it is not possible to have an overview of the optimal or minimum dimensions or distances of the respective combinations for a multiplicity of models with a multiplicity of components and a multiplicity of options. The optimal or minimum dimensions or distances, therefore, cannot be applied or at least cannot always be applied.

For this reason, currently, when planning rooms, blanket minimum distances, measured from the outside of the cladding or covering, of an outer ceiling are used. This blanket minimum distance is not necessarily the actual minimum distance for a specific model or a certain component used. Even in the case of developments and new developments, the previously used blanket minimum distance is adopted and not adjusted. This means that a blanket minimum distance, which corresponds to a maximum minimum distance of all models, is used. This maximum minimum distance is an empirical value that has no correlation with the particular medical floor-mounted device and its components. As a result, space which could otherwise be used is wasted. In addition, a specific medical floor-mounted device may not be installed in a room because a blanket minimum distance means that the medical floor-mounted device is considered unsuitable and/or too large, even though the specific model of the medical floor-mounted device desired has a smaller minimum distance. In this case, small dimensions, for example, one or two centimeters, may often be decisive.

In addition, there are medical floor-mounted devices that are mobile and, for example, can be installed in trucks. As a result of the reduced space, numerous components must be positioned differently, certain dimensions and distances also having to be observed here.

In addition, there are the requirements for the transport of medical floor-mounted devices, for example, in shipping crates, or on transport pallets. Here too, there are a multiplicity of dimensions and distances which are relevant to transport, but which are not given adequate consideration.

### SUMMARY

The aspects of the disclosure address one or more of the disadvantages described above.

It may be regarded as an object of the present disclosure to provide a method for a medical floor-mounted device that overcomes or reduces these disadvantages.

According to one aspect of the present disclosure, a computer-implemented method for a medical floor-mounted device with the following steps is provided: a) provision of a digital model of a medical floor-mounted device, the digital model comprising system information of the medical floor-mounted device which comprises at least component information regarding system components of the medical floor-mounted device; and at least one item of object information comprising at least one spatial requirement of a system component of the medical floor-mounted device, the at least one item of object information being linked to at least one item of system information; and b) derivation from the digital model of minimum distances, in particular three-dimensional minimum distances, which must be taken into consideration when installing the medical floor-mounted device.

According to one aspect of the present disclosure, a computer program is provided. A computer program that contains program code sections that cause a computer to carry out a method for a medical floor-mounted device when the computer program is executed on the computer.

According to one aspect of the present disclosure, a digital storage medium is provided on which a computer program is stored, containing program code sections that cause a computer to carry out a method for a medical floor-mounted device when the computer program is executed on the computer.

A “medical floor-mounted device” is a device that can be used for a medical therapy, medical application, or for a medical examination, in particular, an imaging examination. The term is to be understood broadly in this context and encompasses all kinds of medical devices, in particular, larger medical devices. In particular, a medical floor-mounted device is understood to be a device that can stand independently on a floor, e.g., with a footprint of more than 0.5 m², in particular with a footprint of more than 1 m² to 4 m². A medical floor-mounted device is particularly suitable for accommodating a patient wholly or partially in a hollow space, e.g., a patient tunnel, in order to perform an examination or a treatment. A medical floor-mounted device can be an independent individual device. It can have a wired or wireless connection to other devices. A medical floor-mounted device can preferably be a medical imaging device, in particular a computer tomography device (CT device), an MRI device, a Positron Emission Tomography device (PET device), a PET/MR device, or a Single Photon Emission Computed Tomography CT device (SPECT-CT device).

With the method according to the disclosure, the requirements for room planning during installation of a medical floor-mounted device can be automated and derived as a function of the configuration, i.e., the respective system components. This permits significantly better spatial planning. Furthermore, the method provides information for a data sheet on the minimum spatial requirements for the installation of a medical floor-mounted device.

The method according to the disclosure can also be used for the development of the medical floor-mounted device, i.e., the development of newer models. It allows developers to immediately recognize the effects of changes to system components on spatial requirements. If, for example, a development means that the device would no longer fit on a transport pallet, for example, for air transport, this can be identified at an early stage and countermeasures taken.

The method according to the disclosure for a medical floor-mounted device can take into account every aspect of an operation, service, maintenance, repair, replacement, or renewal of a medical floor-mounted device or a component of a medical floor-mounted device. Furthermore, the method can take into consideration a development or a further development of a medical floor-mounted device or a component of a medical floor-mounted device. This can include the transport of a medical floor-mounted device or a component of a medical floor-mounted device. The operation of a medical floor-mounted device relates to the normal clinical use of a medical floor-mounted device or a component of a medical floor-mounted device. A service relates to any kind of activity not meant by the terms operation, maintenance, repair, replacement, or renewal. For example, this can relate to the loan of a medical floor-mounted device or a component of a medical floor-mounted device. For example, this can relate to the testing of software for a medical floor-mounted device or a component of a medical floor-mounted device. Maintenance relates to all measures of preventive maintenance. Repair relates to all measures for restoring the functionality of a medical floor-mounted device. Replacement relates to the removal and replacement of a medical floor-mounted device or a component of a medical floor-mounted device. Renewal relates to improvement of the condition of at least one element or at least one component of a medical floor-mounted device.

A “digital model” is to be understood broadly within the scope of the present disclosure and includes any possible model of a medical floor-mounted device in the form of a digital representation. A digital model within the scope of the present disclosure can be part of a so-called digital twin. A digital model may comprise data and/or information pertaining to a medical floor-mounted device. In particular, the digital model may comprise digital data regarding the type and the spatial arrangement of the system components from which the medical floor-mounted device is constructed. For example, the digital model may comprise a Computer Aided Design (CAD) model of the medical floor-mounted device.

The term provision is to be understood broadly within the scope of the present disclosure and includes all meanings of the term. In particular, provision may mean that the digital model is retrieved from a data storage device, e.g., from a hard disk or another data storage device, e.g., a remote storage device or a cloud. Generally speaking, provision can also mean that a user inputs information or data. Finally, provision may also include the generation of information or data, e.g., the creation of the digital model.

“System information” pertaining to a medical floor-mounted device may include all information about a particular medical floor-mounted device. This includes, in particular, information about the system components and/or system properties of the floor-mounted device, i.e., the components of which the medical floor-mounted device is composed. The term “system component” is to be understood broadly within the scope of the present disclosure and includes all possible components that may be included in a medical floor-mounted device. The term system property is to be understood broadly within the scope of the present disclosure and includes information regarding properties of a system that do not relate specifically to a system component or only thereto. For example, a mode of operation can be understood as a system property. In the scope of this text, aspects described in relation to one of the terms system component and system property are also applicable to the other of these terms, even if this is not explicitly mentioned.

A system component can be, for example, an emission device, a detector, an electronic device, an input device, a cover, a coil, a magnet, a measuring instrument, or a table for accommodating a patient. In the case of MRI devices, the system component may comprise, for example, a main magnet, a Radio Frequency Power Amplifier (RFPA), a cold head, a table for accommodating a patient, and/or a gradient coil. System information is any information relating to at least one system component. System information preferably relates to geometric properties of a system component, for example, dimensions in one, two, or three dimensions, and/or its position within a medical floor-mounted device. System information may relate to other properties of a system component, for example, weight, model type, configuration, service life, maintenance terms, software, resolution, resistance, maximum voltage, maximum current, service life, tools required for operation, for service, for maintenance, for repair, for replacement, or for renewal, etc.

For example, there may be numerous modes of operation for each different area of the examination object, such as, for example, a patient. A mode of operation is an item of system information for a medical floor-mounted device. For example, there may be numerous service operations. For example, there may be numerous maintenance operations or repair operations for each system component. A service operation, a maintenance operation, or a repair operation is system information for a medical floor-mounted device. For example, there may be system components or parts thereof that are repaired, replaced, or renewed. A repair, a replacement, or a renewal of a system component or parts thereof is system information. Different tools or devices may be required for this in each case, which may result in different minimum distances.

System information for a medical floor-mounted device comprises at least one such item of information for at least one system component.

“Component information” regarding a system component is one of the aforementioned items of system information with regard to a system component of a medical floor-mounted device.

“Object information” comprises at least one “spatial requirement” for a system component of a medical floor-mounted device. The term spatial requirement is to be understood broadly and includes any geometric or spatial requirement that may be relevant to a medical floor-mounted device. This may initially include relevant distances such as, for example, minimum distances, of a system component at least with regard to operation, service, maintenance, repair, replacement or renewal of a system component of a medical floor-mounted device. These distances may relate to all three spatial dimensions. A spatial requirement may also comprise at least one possible position of a respective system component within a medical floor-mounted device. A spatial requirement may also include features required for the installation of the medical floor-mounted device in the room in which the floor-mounted device is to be used. Features in a room may be, for example at least one opening, for example at least one door, at least one window, at least one opening to a ventilation shaft, at least one opening to a shaft, at least one opening for a power socket, at least one opening for connections such as for data transmission, water, fluid, for example a coolant or cooling agent, etc. A spatial requirement may also relate to radiation or exposure to radiation, for example, radioactive, electrical, and/or magnetic radiation. It may be necessary to implement a certain minimum distance or shielding measures. A spatial requirement may also relate to safety requirements. Regardless of any mechanically necessary minimum distance, a safety distance from a medical floor-mounted device or a system component of a medical floor-mounted device may be necessary. These requirements may depend on legal regulations. These requirements may be based on practical considerations, such as, for example, the stature of the user and/or the patient. These requirements may vary over time. A spatial requirement may relate to the transport and logistics of a medical floor-mounted device. The dimensions of a system have an impact on how a medical floor-mounted device can be transported. This may include the type and size of pallet, of protective enclosure, and of shipping container, as well as the type and size of transport vehicle.

An example of a system component is a table for accommodating a patient, also referred to as a patient table. There are various models of patient tables that can be implemented for each medical floor-mounted device. Depending on which model of a patient table is implemented in which model of a medical floor-mounted device, this has repercussions for the spatial requirement of a medical floor-mounted device. For example, at least one spatial requirement arises from the system component comprising a patient table. For example, a patient table must be moved during operation, for example, to accommodate the patient or to position the patient. In doing so, a patient table is moved out of a medical floor-mounted device at least temporarily and partially, and in the process, protrudes at least temporarily and partially beyond the dimensions of a medical floor-mounted device. The extent to which a patient table is moved out depends on numerous factors, such as, for example, the mode of operation, the area of a patient’s body to be examined, and/or the wishes of the examining physician.

For example, the maintenance of a patient table results in at least one spatial requirement. During the maintenance of a patient table, for example, the functionality of a patient table may be examined. In the process, a patient table may be moved out to at least one maximum extension position, which is not reached during normal operation when examining a patient. This results in a different spatial requirement for a patient table than during operation. A maximum extension position is dependent, for example, on the model of a patient table and the model of a medical floor-mounted device.

An example of a system component is a Radio Frequency Power Amplifier (RFPA) of an MRI device. Here, there are also various models, and one or more RFPA can be integrated at different positions in a medical floor-mounted device. For example, the maintenance or repair of an RFPA results in at least one spatial requirement. During maintenance or repair, external devices and tools may be required, which, for example, may be temporarily mounted on a medical floor-mounted device or in the immediate vicinity, at least within 1 m of a medical floor-mounted device. At least a minimum distance is needed as a spatial requirement for assembly and/or for maintenance or repair. An example of an external device and tool is a crane, which is installed on the medical device and has a spatial g. A medical device must be spaced away from the wall in such a manner that such maintenance, repairs, installations, etc., are possible.

An example of a system component is a coil, in particular a gradient coil. For example, when replacing a gradient coil, at least one spatial requirement arises. When replacing a gradient coil, for example, external devices and tools may be required, which, for example, are temporarily mounted on a medical floor-mounted device or in the immediate vicinity, at least within 1 m of a medical floor-mounted device. For a replacement, at least a minimum distance is needed as a spatial requirement. An example of an external device and tool is at least one stand, which must be positioned in front of and behind a medical floor-mounted device. A stand can have a spatial extent on the floor of a room of 1 m x 0.8 m, for example.

An example of a system component is a cold head. A cold head is needed to cool the refrigerant, in particular the liquid helium, which is necessary for the superconducting main magnet of an MRI device. A cold head can be a plate-like and/or square and/or rectangular element, which can have a longitudinal extension of approximately 1 m. An example of a spatial requirement for the replacement of a cold head is when a cold head is pulled upward in the direction of the ceiling of the room from a medical floor-mounted device. This requires a crane and a corresponding distance between a medical floor-mounted device and the ceiling of a room.

These are a few examples that illustrate the scope, amount, and complexity of system information and associated object information.

An example of object information is the respective minimum distance in all three spatial dimensions in a positive and in a negative direction. The respective minimum distance depends on the respective system component and its position in a medical floor-mounted device and varies individually. There are respective minimum distances in all three spatial dimensions for the operation, service, maintenance, repair, replacement, or renewal of the respective system component in the corresponding aspect at the respective position.

These are a few examples that illustrate the scope, amount, and complexity of object information.

The term “link” is to be understood broadly within the scope of the present disclosure and includes all meanings of the term. Within the scope of the present disclosure, the term link can be understood to mean that there is a connection between at least one item of object information and at least one item of system information. If, for example, an item of system information is, for example, a model type of a system component, then an item of object information can be at least a minimum distance in at least one spatial direction, for example with regard to a certain repair. The method according to the disclosure links a corresponding item of object information to a corresponding item of system information. The term link can mean a causal correlation between an item of system information and an item of object information. As described above, such a link has not existed hitherto.

As a result of such a link between at least one item of object information and at least one item of system information, individual properties of system components in a medical floor-mounted device can be taken into consideration. This makes it possible to use individual properties of system components in the method according to the disclosure for a medical floor-mounted device. These individual properties of at least one system component within a digital model can then be used to derive a minimum distance for a medical floor-mounted device. A minimum distance relates to the installation of a medical floor-mounted device in a room in relation to the walls, to the ceiling, to the floor, and to other features of the room. A minimum distance may relate to one, two, or three dimensions in a positive and/or in a negative direction. A minimum distance preferably involves a three-dimensional minimum distance, as both a medical floor-mounted device and a room in which a medical floor-mounted device can be installed are three-dimensional in design. The minimum distance comprises, in particular, a minimum distance in relation to the side walls of a room in which the device is to be installed.

The term installation is to be understood broadly within the scope of the present disclosure and includes all meanings of the term. The term installation may include temporary, stationary, or permanent positioning of a medical floor-mounted device.

The term “derivation” is to be understood broadly within the scope of the present disclosure and includes all meanings of the term. The term derivation from a digital model can mean a causal correlation or a link between a digital model and a derived minimum distance. Minimum distances can be derived from a digital model in an automated manner. In particular, the minimum distances can be calculated from the digital model. If, for example, the individual system components of the floor-mounted device are linked to certain spatial requirements and the digital model includes the spatial arrangement of the system components, the greatest spatial requirement of the individual system components can be derived as the minimum distance. However, it is also possible that the minimum distances in all spatial directions are already included in the digital model and can be read out, i.e., derived, without further calculations. The minimum distances can be three-dimensional (3D), i.e., they contain information about the required minimum distance of the walls and floors from the floor-mounted device in all three spatial directions. Alternatively, the minimum distances can also be only two-dimensional (2D), i.e., they are only output at stand level (floor level). Finally, it is also conceivable to derive only a minimum distance in one dimension, that is to say, for example, forward and/or backward or to one or both sides.

The derived minimum distances are preferably used to create a specification for the medical floor-mounted device, for example, a data sheet or Planning Guide for the medical floor-mounted device. This replaces the empirical values used hitherto with actual minimum distances.

According to one aspect of the present disclosure, the method according to the disclosure can also provide consequences of changes to a medical floor-mounted device in an automated manner. Many components of the medical floor-mounted device can or must be replaced, renewed, or repaired in the course of time. Different or newer models of components, different or newer options of components, and/or developments of components can be used for this purpose. These different or newer models, different or newer options, and/or developments may have different dimensions and/or different distances, which may have an impact on a medical floor-mounted device or a system as a whole, or on minimum distances. Such different dimensions and/or different distances can be taken into consideration in the method according to the disclosure. For example, the minimum distances of a new or different system component can be integrated into a digital model of a medical floor-mounted device as system information in the form of component information for the system component. For example, prior to replacement, renewal, or repair, this enables the method according to the disclosure to be used to ascertain which models can be integrated into an existing medical floor-mounted device, which is positioned in a room.

According to one aspect of the present disclosure, a method is provided for the automated generation of general and customer-specific mechanical requirements for the surrounding area of medical floor-mounted devices.

Requirements may be system type-specific requirements. Such requirements are relevant, in particular, in the product development of a new product. Such requirements may be dependent on the system type and the respective configuration. In this case, the minimum distances as described above must be observed. Minimum distances may be necessary for various reasons, as described above. This may be the extension and retraction of a system component. This may be the removal of a system component. This may be a repair, a replacement, or a renewal of a system component. This may be the transport of a system component or a medical floor-mounted device with a system component. This may be the use of additional tools, devices, gantries, etc., around a medical floor-mounted device, which are necessary or helpful for operation, service, maintenance, repair, replacement, or renewal.

System information and/or object information can be stored in a database. System information and/or object information can be stored in a database in correlation or in a causal relationship. System information and/or object information can be updated and/or adjusted automatically and/or dynamically. This results in a feedback loop between system information and object information. System information and/or object information can be entered in a database from different sources. A source can be a manufacturer or a user of a medical floor-mounted device or a system component, which enter the corresponding system information and/or object information in a database. A database can be updated at regular intervals, for example, by a manufacturer or a user. A database can be stored on a computer-readable medium or a computer. A database can be stored in a network, such as, for example, a cloud.

Requirements may be instance-specific requirements. In particular, such requirements depend on special combinations and configurations of users. A user who has or would like to install a medical floor-mounted device has customer-specific criteria for the medical floor-mounted device, for example, depending on the use, such as the measurements to be performed. A user who has or would like to install a medical floor-mounted device has customer-specific spatial conditions that should be taken into account. A spatial condition is, for example, the space or the area that a customer has available for a medical floor-mounted device. There are situations in which the spatial conditions do not enable a medical device to be installed without modifications to the medical device or the spatial conditions. In such cases, a minimum requirement for structural alterations is derived as a function of the spatial conditions and the customer-specific criteria. Customer-specific criteria, customer-specific spatial conditions, and a minimum requirement for structural alterations are examples of instance-specific requirements that are taken into account within the scope of the method according to the disclosure.

Structural alterations or structural measures may involve a multiplicity of changes. For example, moving a wall, a ceiling, or a floor is a structural alteration. For example, changing the position of one of the openings described herein is a structural alteration. For example, the position of a door or a shaft can be altered, or a door or a shaft can be an integrated in a different position in a room. A structural alteration may, for example, be a particular protective measure to shield against radiation such as, for example, X-rays or fields such as, for example, magnetic fields. A structural alteration may, for example, be an alteration in the thickness of a wall or the at least partial movement of a wall or an alteration of the dimensions of a wall projection.

Further features and advantages will emerge from the dependent claims.

According to one aspect of the present disclosure, the method comprises the steps of provision of modified system information or modified object information, adjustment of the digital model based on the modified system information or the modified object information, and derivation of modified three-dimensional minimum distances.

The term “adjustment” is to be understood broadly within the scope of the present disclosure. In particular, it means that the digital model is modified according to the modified system information and/or modified object information. At least one item of system information or at least one item of object information may be subject to change. For example, a system component may be modified as part of the configuration of an associated medical floor-mounted device. An alteration may be the integration of an additional or different option. An alteration may also be a repair, a replacement, or a renewal of a system component. Finally, a system component can also be altered within the scope of a development of the medical floor-mounted device. For example, the geometry and/or the weight of a system component may change. Such a change can have an effect on object information and/or on system information in connection with a system component. According to the present disclosure, such a change can be taken into consideration and lead to a change or adjustment of a digital model. In other words, a change of object information and/or system information leads to a causal and/or correlated and/or automatic adjustment of a digital model, for example.

For example, during the product development of a medical floor-mounted device, numerous models of numerous system components can be implemented in a respective medical floor-mounted device within a digital model. According to the present disclosure, the minimum distances of a medical floor-mounted device are adjusted in each case and may be derived in the current form.

According to one aspect of the present disclosure, a computer-implemented method is provided, an item of object information comprising at least one item of component information about an operation, service, maintenance, repair, replacement, or renewal procedure of the system component associated with the item of object information.

The terms operation, service, maintenance, repair, exchange, or renewal procedure are to be understood as explained above. According to the present disclosure, object information may comprise information regarding a corresponding procedure of a corresponding system component. In particular, the object information comprises a spatial requirement associated with the procedure. This may arise, for example, as a result of an external device such as a crane being required for the procedure, which has a certain spatial extent. Furthermore, a spatial requirement may arise as a result of a system component having to be completely removed from the medical floor-mounted device during replacement, so that it protrudes significantly beyond the device.

For example, the system component patient table results in at least one spatial requirement. For example, a patient table must be moved during operation, for example, to accommodate the patient or to position the patient. In the process, a patient table is moved out of a medical floor-mounted device at least temporarily and partially, and in the process protrudes at least temporarily and partially beyond the dimensions of a medical floor-mounted device. How far a patient table is extended depends on numerous factors, such as, for example, the mode of operation, the area of a patient’s body to be examined, and/or the wishes of the examining physician. Component information is, for example, the model of a patient table. Here, a specific model of a patient table in a specific medical floor-mounted device in a specific mode, for example, during operation, leads to a specific spatial requirement for the system component of a patient table as object information.

According to one aspect of the present disclosure, a computer-implemented method is provided which also comprises the step of compiling at least one item of object information in the form of requisite minimum distances which are necessary to be able to perform operation, service, maintenance, repair, replacement or renewal procedures on system components, and a step of adjusting the digital model on the basis of the at least one item of object information compiled.

The term compilation is to be understood broadly in the context of the present disclosure and includes all meanings of the term. A user can perform compilation. It can also take place in a partially automated manner. In particular, this means that for at least one system component, the relevant mechanical and/or geometric requirements, in particular the dimensions, as well as the spatial requirements for operation, service, maintenance, repair, replacement, or renewal procedures, are measured or calculated. A user can do this. These are then entered into the digital model as object information.

According to one aspect of the present disclosure, a computer-implemented method is provided, in which three-dimensional minimum distances are derived in the form of an overall envelope.

Three-dimensional minimum distances can be derived as individual points in space at a corresponding minimum distance from a system component in three spatial dimensions. This results in values for the three spatial dimensions, for example, in a coordinate system with an x, y, and z axis, for example, with regard to a system component. If a medical floor-mounted device or a system component of a medical floor-mounted device changes its position in the room over time, the individual points in space of the minimum distances also change, and arrays of curves are formed. A curve that envelopes this array of curves is an envelope curve.

An envelope can be derived for these values of the minimum distances of at least one system component in three spatial dimensions. Multiple envelopes can be derived for multiple three-dimensional minimum distances of at least one system component or three-dimensional minimum distances of multiple system components. A curve that envelops these envelopes is an overall envelope.

The terms envelope and overall envelope are understood here as defined in the fields of mathematics and/or physics.

If there is more than one envelope, these can be combined to form an overall envelope. An overall envelope can thus comprise at least one envelope. A total minimum distance is then derived from an overall envelope.

According to one aspect of the present disclosure, a computer-implemented method is provided, at least one item of object information comprising at least one trajectory or at least one envelope of a trajectory.

The dimensions and distances for operation, service, maintenance, repair, replacement, or other relevant events can take into account not only a one or two-dimensional distance, but a respective distance in all three spatial dimensions. It is possible for a medical floor-mounted device or a system component of a medical floor-mounted device to move in at least one direction. The minimum distance of a medical floor-mounted device or a system component of a medical floor-mounted device then also alters in at least one direction. Owing to the three spatial dimensions, this results in a spatial curve, which can be described by a trajectory. It can be a one, two, or three-dimensional trajectory. Such a trajectory must be taken into account for each component, each option of each component, and each possible position of each component. Based on a trajectory associated with at least one system component, an envelope of a trajectory can be determined. The terms trajectory and envelope are understood here as defined in the fields of mathematics and/or physics. Thus, a trajectory or an envelope can be three-dimensional.

A method according to the disclosure may comprise multiple items of object information. Multiple items of object information may originate from one system component or from multiple system components. At least one item of object information may comprise a trajectory or an envelope. At least one item of object information may comprise multiple trajectories or multiple envelopes. If there is more than one trajectory or more than one envelope, these can be combined into an overall trajectory or an overall envelope. Thus, an overall trajectory may comprise at least one trajectory, or an overall envelope may comprise at least one envelope.

Alternatively, an overall trajectory may comprise all trajectories, or an overall envelope may comprise all envelopes.

According to one aspect of the present disclosure, a computer-implemented method is provided, the derivation of the three-dimensional minimum distances being performed using an optimization function of the digital model.

A three-dimensional minimum distance involves a multiplicity of distances or at least multiple distances in at least one spatial dimension. A three-dimensional minimum distance, therefore, comprises a multiplicity of information. A multiplicity of three-dimensional minimum distances results in an even larger multiplicity of information. When deriving at least one three-dimensional minimum distance, it is possible to use a multiplicity of information through optimization or using an optimization function.

A multiplicity of information can be regarded as a multiplicity of limitations for which one or more permissible solutions are to be found. This can be understood as solving an optimization problem in the sense of mathematical optimization. Thus, an optimization function can be used, which can lead to a derivation of at least one, in particular three-dimensional, minimum distance. A multiplicity of limitations can be understood as a linear optimization problem.

According to one aspect of the present disclosure, a computer-implemented method is provided, further comprising the steps of d) provision of a room model of a room in which the medical floor-mounted device is to be installed; and e) performance of an optimization function of the digital model and the room model to find out whether the medical floor-mounted device can be installed in the room while observing the minimum distances, and if not, making of suggestions for structural alterations to the room in which the medical floor-mounted device is to be installed, and/or making of suggestions for alterations to system components of the medical floor-mounted device.

A medical floor-mounted device can be positioned in a room. Each room has properties and features, such as dimensions in the form of height, width, and length, or openings for doors, windows, shafts, sockets, or other connections, for example. Such properties and features can be implemented in a room model of a room. In one aspect of the present disclosure, such a room model can be provided for a room in which a medical floor-mounted device is to be installed and/or positioned.

In addition, a digital model as described above is provided, and three-dimensional minimum distances are derived. As described above, an optimization function can be used to derive three-dimensional minimum distances. It is possible to use information about properties and features of a room, for example, in the form of a room model of a room. An optimization function can use a digital model of a medical floor-mounted device and a room model of a room. This makes it possible to determine whether a medical floor-mounted device in its specific configuration and the minimum distances derived therefrom can be installed in a room using a room model provided. The aspect, according to the disclosure, can take into account all information from all system components of the respective model in the respective option and their combination. The aspect, according to the disclosure, can determine an existing additional distance if the spatial dimensions of the room are larger than the space requirement of a medical floor-mounted device, including all minimum distances, or can determine an additional distance required if the spatial dimensions of the room are smaller than the space requirement of a medical floor-mounted device, including all minimum distances.

If a method of the aspect according to the disclosure concludes that a medical floor-mounted device cannot be installed in a room, the method according to the disclosure can make suggestions as to how it might be possible to install a medical floor-mounted device in a room. The method, according to the disclosure, can consider ways in which a corresponding room can be altered. For example, it may be helpful to remove one or more of the openings mentioned above or to modify a room in such a manner that an opening does not need to be used. For example, a new ventilation shaft, a new socket, or a new door could be constructed in a different position. The method, according to the disclosure, can, in addition or alternatively, consider ways in which a corresponding medical floor-mounted device can be altered such that it can be installed in the corresponding room. For example, one or more system components of a medical floor-mounted device could use a different model in a specific option, which has a shorter minimum distance in a specific spatial dimension, for example, a smaller patient table or a patient table with a shorter travel path, as a result of which the minimum distance of a medical floor-mounted device can be shorter.

According to one aspect of the present disclosure, a computer-implemented method is provided, the optimization function applying a simplex method.

A simplex method or simplex algorithm is a numerical optimization method for solving linear optimization problems. It solves such a problem exactly after a finite number of steps or determines its insolubility or unlimited nature. The basic idea of the simplex method was developed by George Dantzig in 1947.

According to one aspect of the present disclosure, a computer-implemented method is provided, it being possible for the medical floor-mounted device to be a medical imaging device, in particular a CT device, an MRI device, a PET device, a PET/MR device, or a SPECT-CT device.

As described above, a medical floor-mounted device may be a medical imaging device. Such medical imaging devices comprise numerous system components that have corresponding space requirements and minimum distances.

According to one aspect of the present disclosure, a computer-implemented method is provided, the medical floor-mounted device being an MRI device, and object information being at least a minimum distance in at least one spatial direction for the operation, service, maintenance, repair or replacement of a system component, or at least spatial information about the room in which the medical floor-mounted device is to be installed, for example positions of openings such as doors, windows, sockets, ventilation shafts, filter openings or positions of cables and connections.

As described above, a medical floor-mounted device may be a medical imaging device and, in particular, an MRI device.

As described above, object information may include at least a minimum distance in at least one spatial direction. A minimum distance in at least one spatial direction may relate to the operation, service, maintenance, repair, or replacement of a system component. The minimum distances for the various aspects differ for a system component as described above. As described above, object information may include at least one item of information about the room in which the medical floor-mounted device is to be installed. As described above, information about the room may include, for example, positions of openings such as doors, windows, sockets, ventilation shafts, filter openings, or positions of cables and connections.

According to one aspect of the present disclosure, the method according to the disclosure includes the consideration of requirements for the transport of medical floor-mounted devices, for example, in shipping crates or on transport pallets. Here too, there is a multiplicity of dimensions and distances which are relevant to transport but have not yet been taken into consideration in the prior art.

A spatial requirement may relate to the transport and logistics of a medical floor-mounted device. The dimensions of a system have an impact on how a medical floor-mounted device can be transported. This may include the type and size of a pallet, protective cover, and shipping container, as well as the type and size of a transport vehicle.

The method according to the disclosure can also be used for the development of the medical floor-mounted device, i.e., the development of newer models. It allows developers to immediately recognize the effects of changes to system components on the spatial requirements. If, for example, a development means that the device would no longer fit on a transport pallet, for example, for air transport, this can be recognized at an early stage and countermeasures taken.

According to one aspect of the present disclosure, the method according to the disclosure includes the consideration of requirements of portable or mobile medical floor-mounted devices. There are medical floor-mounted devices that are mobile and can be installed in trucks, for example. As a result of the reduced space, numerous components must be positioned differently, certain dimensions and distances also having to be observed here. Thus, other component information from system components may result in system information, and/or other spatial requirements of a system component may result in object information, which can be taken into consideration in the method according to the disclosure.

According to one aspect of the present disclosure, a computer program is provided. A computer program according to the disclosure comprises, in particular, program code sections which represent the method steps according to the disclosure when a computer program is executed on a computer.

As a result, the method according to the disclosure can be defined and carried out repeatedly, and control can be exercised over the transfer of the method according to the disclosure. The computer program is preferably configured in such a manner that a computing unit can perform the method steps according to the disclosure using the computer program. The program code sections can, in particular, be loaded into a memory of a computing unit and typically executed using a processor of the computing unit with access to the memory. When the computer program, in particular the program code sections, is executed in the computing unit, all aspects of the described method according to the disclosure can typically be performed.

A computer program can be stored on a digital storage medium. The computer program is stored, for example, on a physical, computer-readable storage medium and/or digitally as a data package in a computer network. The computer program can represent the physical, computer-readable storage medium and/or the data package in the computer network. Thus, the disclosure can also originate from the physical, computer-readable storage medium and/or the data package in the computer network. The physical, computer-readable storage medium can usually be connected directly to the computing unit, for example, by inserting the physical, computer-readable storage medium into a DVD drive or plugging it into a USB port, as a result of which the computing unit can access the physical, computer-readable storage medium, in particular for reading. The data package can preferably be retrieved from the computer network. The computer network can comprise the computing unit or be indirectly connected to the computing unit using a Wide Area Network (WAN) or a (Wireless) Local Area Network connection (WLAN or LAN). For example, the computer program can be stored digitally on a cloud server at a storage location on the computer network and transferred to the computing unit using the WAN via the internet and/or using the WLAN or LAN, in particular by retrieving a download link which refers to the storage location of the computer program.

The disclosure is described and explained in more detail hereinafter with reference to the exemplary aspects shown in the figures. In principle, in the following figure description, essentially unchanged structures and units are referred to with the same reference characters as when the respective structure or unit first appeared.

All aspects described herein can be combined with one another, unless explicitly stated otherwise.

### BRIEF DESCRIPTION OF THE DRAWINGS

The properties, features, and advantages of this disclosure described above, as well as the manner in which they are achieved, will become clearer and more understandable in connection with the following description of the exemplary aspects, which are explained in more detail in connection with the figures.

Aspects are described hereinafter with regard to the attached figures.

FIG. 1 shows a diagrammatic flowchart of a method according to an aspect of the present disclosure;

FIG. 2 shows a diagrammatic cross-section of an aspect of the present disclosure; and

FIG. 3 shows a diagrammatic top view of an aspect of the present disclosure.

### DETAILED DESCRIPTION

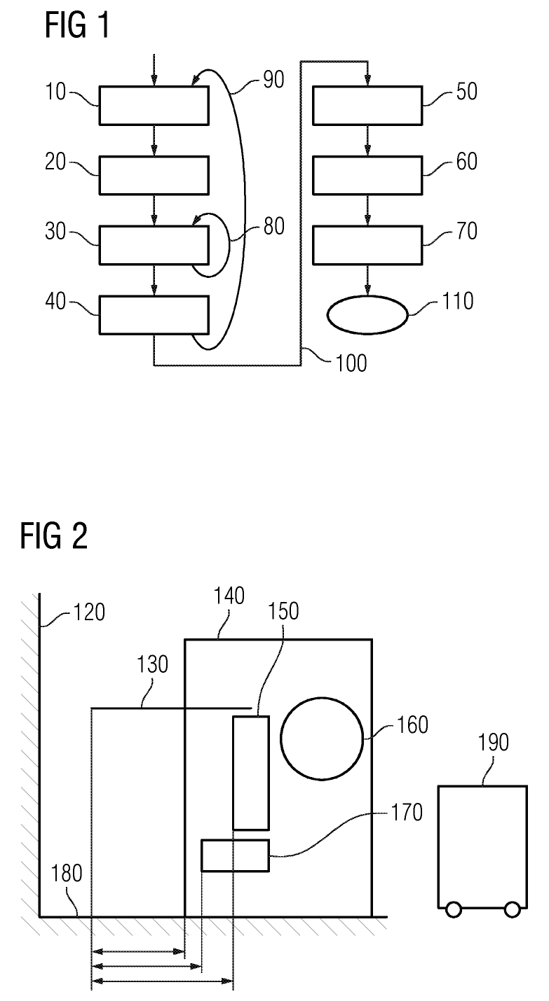

FIG. 1 shows a diagrammatic flowchart of a method according to an aspect of the present disclosure. Not all the steps shown are necessary for all aspects. Individual or multiple steps may be omitted or not be implemented. Individual or multiple steps may be implemented at a different time, i.e., the sequence of the steps can be variably adjusted. Additional steps may also be implemented before or after individually described steps.

System- and instance-specific requirements are described above. The system- and instance-specific requirements shown in FIG. 1 are examples and are not to be interpreted as an exhaustive list. The disclosure comprises all relevant system type-specific and instance-specific requirements, even if they are not shown in FIG. 1 or mentioned in this exemplary aspect. Furthermore, not all of the requirements shown are essential or necessary across all aspects, according to the disclosure.

FIG. 1 shows system-type-specific requirements on the left-hand side, labeled with reference characters 10, 20, 30, and 40. FIG. 1 shows instance-specific requirements on the right-hand side, labeled with the reference characters 50, 60, and 70.

The first step 10 relates to the compilation of requirements, which are described in more detail above. Requirements may be, for example, mechanical requirements, such as wall distances or room height, for example.

The second step 20 relates to the digitization of requirements, for example, relative to system properties and/or system components. In this step, a relationship between the requirements and the system components and/or properties is established and stored during digitization.

The third step 30 relates to the development of a product and a change in system properties and/or system components. As described above, FIG. 1 shows an example of the present disclosure in the context of product development. The various system components and/or system properties can be individually adjusted and modified in the method according to the disclosure. This results in a dynamic adjustment 80 within the scope of the method according to the disclosure, for example, a dynamic adjustment of the minimum spatial requirements for the respective system component, for the respective system property, and for the medical floor-mounted device.

The fourth step 40 relates to the derivation of requirements for the medical floor-mounted device. An overall requirement for the medical floor-mounted device can be derived from the spatial requirements for the respective system component and for the respective system property.

This process, which is described with regard to the four steps e10, 20, 30, 40, can be repeated multiple times 90, for example, during the development of a product, a product component, or a follow-up product.

This process, as described with respect to the four rectangles 10, 20, 30, 40, may have an impact on instance-specific requirements 100 or be related to them 100.

The fifth step 50 relates to the consideration of a certain specific configuration of a medical floor-mounted device, for example, a user configuration. This may be a configuration that is in operation or at the planning stage.

The sixth step 60 relates to the consideration of a certain specific configuration of spatial conditions of a room or an area, for example, the spatial conditions of a room or an area of a user.

The seventh step 70 relates to the derivation of a minimum requirement for structural alterations. As described above, multiple structural alterations may be possible and/or beneficial for a medical floor-mounted device that is installed or to be installed. The method, according to the disclosure, can derive possible structural alterations and the minimum requirements for structural alterations from the specific configuration of a medical floor-mounted device and/or the specific configuration of spatial conditions.

In this way, it is possible to derive the optimal framework conditions for installing a medical floor-mounted device from at least one item of system information and at least one item of object information, and to install a medical floor-mounted device 110.

FIG. 2 shows a diagrammatic cross-section of an aspect of the present disclosure. This aspect relates to the replacement of an RFPA 150. For this purpose, a crane 130 is used as an example of a device or tool, and an RFPA trolley 190 is used as an example of an aid. In this case, a high-frequency receiving component 170 can also be replaced, for example.

Furthermore, FIG. 2 shows a medical floor-mounted device 140 comprising a hollow space 160, an RFPA 150, and a high-frequency receiving component 170. Part of the room is represented by a wall 120 and a floor 180.

The four vertical lines in FIG. 2, below the floor 180, show, from left to right, the distances between the crane 130 and an external wall of a medical floor-mounted device 140, a high-frequency receiving component 170, and an RFPA 150. These three distances differ in each case. For example, the difference can be 1-10 cm in each case, preferably 2-6 cm. The crane 130 can be mounted in a different position with a different extension outside the medical floor-mounted device, depending on whether the distance is taken from an external wall of a medical floor-mounted device 140, an RFPA 150, or a high-frequency receiving component 170. For a replacement of an RFPA 150 or a high-frequency receiving component 170, a crane 130 therefore requires less space from a wall than if an external wall of a medical floor-mounted device 140 were used as a reference point. These distances and the positions within a medical floor-mounted device 140 may vary across different models or developments of an RFPA 150 or a high-frequency receiving component 170. These aspects can be taken into account in accordance with the method disclosed.

In addition, for example, other devices or tools are also required, such as an RFPA trolley 190. For example, this must be positioned at a particular location in the vicinity of a medical floor-mounted device 140 during replacement and has its own spatial requirements.

Such aspects can also be taken into consideration using the method according to the disclosure.

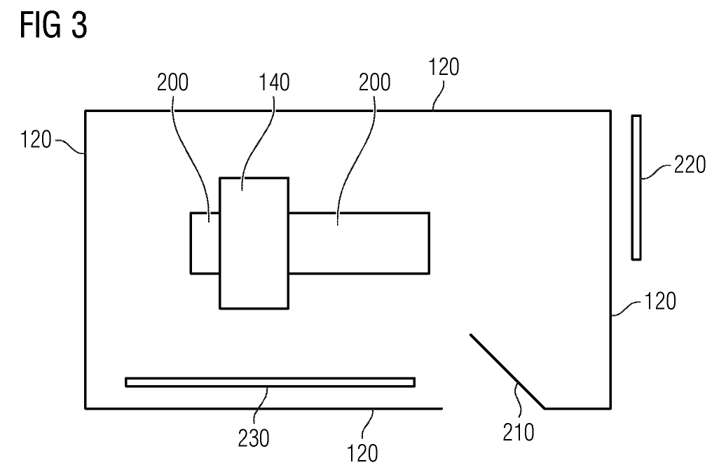

FIG. 3 shows a diagrammatic top view of an aspect of the present disclosure. This aspect relates to the implementation of a medical floor-mounted device in a room and the spatial conditions. FIG. 3 shows a room with walls 120 in which there is a medical floor-mounted device 140 and a patient table 200 that protrudes from the device 140 and can be moved out. For example, an opening 210, such as a door 210, is shown as a possible spatial condition of a room.

As shown in FIG. 2, the corresponding distances must be taken into account, for example, for the replacement or the operation of respective system components. In addition, additional requirements are described for FIG. 3. For example, the positions of openings 210, such as for example, a door 210, must be taken into account as spatial conditions.

A spatial requirement may also relate to radiation or to exposure to radiation, for example, radioactive, electrical, and/or magnetic radiation. It may be necessary to implement a certain minimum distance or shielding measures. A spatial requirement may also relate to safety requirements. Regardless of a mechanically required minimum distance, a safety distance from a medical floor-mounted device or its system component may be required. These requirements may depend on legal regulations.

A structural alteration may, for example, be a protective measure to shield against radiation, such as X-rays or magnetic fields. A structural alteration may, for example, be a change in wall thickness or the moving of a wall.

In the example in FIG. 3, two protective measures 220, 230 are shown by way of example. A protective measure may be shielding using an additional structure made of a suitable material, for example, iron. FIG. 3 shows a protective measure 230 inside the room with the medical floor-mounted device 140 and a protective measure 220 outside the room with the medical floor-mounted device 140. A protective measure 230 inside the room reduces the room's dimensions accordingly. Such a protective measure 220, 230 can, for example, improve the shielding of a magnetic field.

Although the disclosure has been illustrated and described in more detail by the preferred exemplary aspects, the disclosure is not limited by the disclosed examples, and other variations may be derived therefrom by a person skilled in the art without departing from the scope of the disclosure.

The disclosure relates to a computer-implemented method for a medical floor-mounted device, comprising the following steps a) provision of a digital model of a medical floor-mounted device, the digital model comprising system information of the medical floor-mounted device, which comprises at least component information regarding system components of the medical floor-mounted device; and at least one item of object information comprising at least one spatial requirement of a system component of the medical floor-mounted device, the at least one item of object information being associated with at least one item of system information; and b) derivation from the digital model of minimum distances, in particular three-dimensional minimum distances, which must be taken into consideration when installing the medical floor-mounted device.

1. A computer-implemented method for determining installation requirements for a medical floor-mounted device, comprising:
providing a digital model of the medical floor-mounted device, wherein the digital model comprises:
system information of the medical floor-mounted device, which comprises at least component information regarding system components of the medical floor-mounted device; and
at least one item of object information which comprises at least one spatial requirement of a system component of the medical floor-mounted device, wherein the at least one item of object information is associated with at least one item of system information; and
deriving from the digital model of three-dimensional minimum distances, which are taken into consideration when installing the medical floor-mounted device.

2. The method as claimed in claim 1, further comprising:
providing modified system information or modified object information, adjusting the digital model based on the modified system information or the modified object information, and deriving from the adjusted digital model, modified three-dimensional minimum distances.

3. The method as claimed in claim 1, wherein the object information comprises an item of component information about an operation, service, maintenance, repair, replacement, or renewal procedure of the system component associated with the object information.

4. The method as claimed in claim 1, further comprising:
compiling an item of object information in a form of necessary minimum distances required to perform operation, service, maintenance, repair, replacement, or renewal procedures on system components, and adjusting the digital model based on the item of object information compiled.

5. The method as claimed in claim 1, wherein three-dimensional minimum distances are derived in a form of an overall envelope.

6. The method as claimed in claim 1, wherein the item of object information comprises at least one trajectory or at least one envelope of a trajectory.

7. The method as claimed in claim 1, wherein the deriving of the three-dimensional minimum distances is performed using an optimization function of the digital model.

8. The method as claimed in claim 1, further comprising:
providing a room model of a room in which the medical floor-mounted device is to be installed; and
performing an optimization function of the digital model and the room model to find out whether the medical floor-mounted device is installable in compliance with the minimum distances in the room, and if not, making suggestions for structural alterations to the room in which the medical floor-mounted device is to be installed, and/or making suggestions for changes to system components of the medical floor-mounted device.

9. The method as claimed in claim 8, wherein the optimization function uses a simplex method.

10. The method as claimed in claim 1, wherein the medical floor-mounted device is a medical imaging device.

11. The method as claimed in claim 1, wherein the medical floor-mounted device is an MRI device, and wherein an item of object information is at least one minimum distance in at least one spatial direction for operation, service, maintenance, repair, or replacement of a system component, or at least one item of spatial information about a room in which the medical floor-mounted device is to be installed, including positions of openings selected from a group consisting of doors, windows, ventilation shafts, filter openings, and positions of cables, and connections.

12. A non-transitory computer-readable digital storage medium storing a computer program containing program code sections that, when executed by a computer, cause the computer to:
 provide a digital model of a medical floor-mounted device, wherein the digital model comprises:
system information of the medical floor-mounted device including at least component information regarding system components of the medical floor-mounted device, and
at least one item of object information comprising at least one spatial requirement of a system component of the medical floor-mounted device, wherein the at least one item of object information is associated with at least one item of system information; and
derive, from the digital model, three-dimensional minimum distances that must be taken into consideration when installing the medical floor-mounted device.
